(19) United States
(12) Patent Application Publication
(10) Pub. No.: US 2016/0199406 A1
(43) Pub. Date: Jul. 14, 2016

(54) NON-ANESTHETIC PROTECTIVE GASES IN COMBINATION WITH LIQUID ANESTHETIC AGENTS FOR ORGAN PROTECTION

(71) Applicants: Klaus Michael SCHMIDT, Halifax (CA); David Cecil ROACH, Halifax (CA)

(72) Inventors: Klaus Michael SCHMIDT, Halifax (CA); David Cecil ROACH, Halifax (CA)

(21) Appl. No.: 14/912,657

(22) PCT Filed: Aug. 19, 2014
(86) PCT No.: PCT/CA2014/000630

§ 371 Date: Feb. 18, 2016

Related U.S. Application Data
(60) Provisional application No. 61/867,367, filed on Aug. 19, 2013.

(51) Int. Cl. A61K 33/04; A61K 9/00; A61K 31/08; A61K 33/00

(52) U.S. Cl. CPC A61K 33/04; A61K 33/00; A61K 9/0019; A61K 9/007; A61K 31/08

(57) ABSTRACT

A method of providing anesthesia and organ-protection to a subject in need thereof comprises co-administering to the subject a non-anesthetic protective gas in an amount effect to provide organ protection, and a liquid anesthetic agent in an amount effective to provide anesthesia, at normobaric conditions.

### CROSS REFERENCES TO RELATED APPLICATIONS

This application claims priority from U.S. Provisional Patent Application No. 61/867,367, filed on Aug. 19, 2013.

### BACKGROUND

Blood flow to the organs may be compromised in a variety of surgical and non-surgical conditions and procedures. Ischemic complications including death may occur.

The satisfactory treatment of, for example, traumatic brain injury (TBI) and/or spinal cord injury represents a major unmet clinical need. It has been estimated that each year, in the United States alone, approximately 1.5 million people will sustain TBI. At least 15% will be hospitalized and 3% will die. For approximately 80,000 of those that are hospitalized and survive, the injury will herald the onset of long-term disability. A significant number of those that are injured but are not admitted to hospital are also likely to suffer significant health care problems.

One of the difficulties in developing strategies to treat brain or spinal cord injury is the highly heterogeneous nature of both the initial injury and its subsequent pathological development. Severe life-threatening head trauma will inevitably involve mechanisms of developing injury, which differ from those that occur following a mild contusion. Nonetheless, a number of common neuronal and biochemical mechanisms are thought to be involved. It seems to be generally agreed that, while the primary injury will cause immediate mechanical damage to both the vasculature and to neuronal tissue, there follows a complex series of interacting injury cascades driven by, among other things, ischemia, cerebral and spinal chord edema and axotomy. The fact that these processes lead to a developing “secondary” injury has given some hope that interventions can be devised which arrest the development of injury or minimize its impact. Similar mechanisms are described for ischemia of other organs with devastating effects.

### SUMMARY OF THE DISCLOSURE

The following summary is provided to introduce the reader to the more detailed discussion to follow. The summary is not intended to limit or define the claims.

With the growing importance of protective strategies, the following disclosure relates to the protection of organs in humans and animals from the side effects of modern liquid anesthetic agents, and/or the devastating effects of glucose and/or oxygen deprivation. Certain side effects of liquid anesthetic agents are discussed in Tang et al., 2013; Kundra et al., 2011; Regueiro-Purrinos et al., 2011; Tetrault et al., 2008; Hu et al., 2014; Liu et al, 2010; and Tong et al., 2013.

Delivering anesthesia is based on delivering at least one of sedation, sleep and pain control. Anesthetics can be gaseous (e.g. xenon and/or nitrous oxide) or liquid (i.e. buprenorphine, propofol, barbiturates, sevoflurane, isoflurane, desflurane). The liquid anesthetic agents can be in the form of a solution, emulsion or any other liquid composition. The liquid anesthetic agents can be delivered orally, directly into the blood stream, or by inhalation. To administer such liquid anesthetic agents by inhalation, they are vaporized. The subset of liquid anesthetic agents that are vaporized to be administered by inhalation are called anesthetic vapors.

The present application relates to the synergistic application of protective gases that provide no anesthetic effects at atmospheric pressure (referred to herein as non-anesthetic protective gases), such as argon and hydrogen sulfide, and liquid anesthetic agents, where the organs are protected by the non-anesthetic protective gases, and the liquid anesthetic agents provide the anesthetic component absent in the non-anesthetic protective gases.

The non-anesthetic protective gases may provide some protection against the side effects of the liquid anesthetic agents, and also against the harmful effects of oxygen and glucose deprivation.

According to one aspect, a method of providing anesthesia and organ-protection to a subject in need thereof comprises co-administering to the subject a non-anesthetic protective gas in an amount effect to provide organ protection, and a liquid anesthetic agent in an amount effective to provide anesthesia, at normobaric conditions.

According to another aspect, a use is provided for a non-anesthetic protective gas in combination with a liquid anesthetic agent, at normobaric conditions. The anesthetic protective gas in combination with the liquid anesthetic agent is for providing anesthesia and organ-protection to a subject in need thereof.

According to another aspect, a method of providing organ-protection to a subject in need thereof comprises co-administering to the subject a non-anesthetic protective gas and a liquid anesthetic agent, in amounts effective to provide organ protection, at normobaric conditions.

In some examples, the non-anesthetic protective gas may be helium, neon, argon, krypton, or radon. In some particular examples, the non-anesthetic protective gas may be argon. The argon may optionally be administered at a concentration of between 1% and 79%.

In some examples, the non-anesthetic protective gas may be hydrogen sulfide. The hydrogen sulfide may be administered at a level of less than 1000 ppm.

In some examples, the liquid anesthetic agent may be administered in liquid form. For example the liquid anesthetic agent may be administered intravenously. In some such examples, the liquid anesthetic agent may be thiopental, propofol, ketamine, hypnomidate, barbiturate, buprenorphine, or dexmedetomidine.

In some examples, the liquid anesthetic agent may be administered in a vapor state. In some such examples, the liquid anesthetic agent may be sevoflurane, isoflurane or desflurane. In some particular examples, the liquid anesthetic agent may be sevoflurane.

In some examples, the liquid anesthetic agent may be administered locally to achieve local or regional anesthesia.

In some examples, the non-anesthetic protective gas and the liquid anesthetic agent may be administered to the subject by a oxygenator in a heart-lung machine, a membrane based device, and/or an extracorporeal membrane oxygenation (ECMO) setup.

In some examples, the non-anesthetic protective gas and the liquid anesthetic agent may be administered to the subject by a ventilator.

In some examples, the ventilator may comprise one or more gas separation membranes that selectively retains, sequesters or exhausts the non-anesthetic protective gas from the air exhaled by the subject, and optionally i) when the non-anesthetic protective gas is retained it is available for further use on the patient and ii) when the non-anesthetic protective gas is sequestered or exhausted, it is subsequently recaptured for future use.

In some examples, administration of the non-anesthetic protective gas in combination with the liquid anesthetic agent may reduce damage to the brain, the spinal cord, the kidney, the liver and/or the heart.

In some examples, administration of the non-anesthetic protective gas in combination with the liquid anesthetic agent may reduce damage resulting from hypoxemia, hypoxia, ischemia, cerebral edema or axotomy.

In some examples, the non-anesthetic protective gas and the liquid anesthetic agent may be administered concurrently or sequentially.

In some examples, the liquid anesthetic agent and non-anesthetic protective gas may be administered to a patient that is undergoing surgery, optionally general surgery or trauma surgery, optionally trauma surgery to treat impact trauma, such as traumatic CNS injury (brain injury or spinal cord injury) or traumatic injury to organs in the torso.

In some examples, the non-anesthetic protective gas and the liquid anesthetic agent may be administered in the absence of xenon.

In some examples, the non-anesthetic protective gas and the liquid anesthetic agent may be administered in the absence of nitrous oxide.

In some examples, the non-anesthetic protective gas and liquid anesthetic agent are administered under normothermic conditions.

These and other objects, advantages, and features of the disclosure will become apparent to those skilled in the art as more fully described below.

### BRIEF DESCRIPTION OF DRAWINGS

The drawings included herewith are for illustrating various examples of articles, methods, and apparatuses of the present specification and are not intended to limit the scope of what is taught in any way. In the drawings:

FIG. 1 is a graph showing the experimental design matrix of the Examples section below, including a summary of the number of mice (n) used in each condition.

FIG. 2 (Top) is a summary of data derived from 24, 48 and 72 hour post-injury analysis. Means and standard errors of difference scores (uninjured—injured numbers of annexin V labeled cells within the YFP population) are plotted. At 24 hours post-injury, early onset of injury is indicated in control mice, as revealed by negative difference scores. Peak neuroprotection by argon is observed at 48 hours, and is retained at 72 hours. (Bottom) Schematic summary describing results for Argon neuroprotection in the context of RGC response to injury. During early induction of apoptosis, the number of RGCs that are responsive to annexin V binding is at its highest, and declines as the apoptotic cascade progresses. Early phase difference scores in injured retinas reflect an increase in annexin V labeling during apoptotic induction. As cells progress through apoptosis, they lose their ability to bind annexin V, thus reducing the pool of annexin V-sensitive cells.

FIG. 3 (Top) is a descriptive statistics table for the data described in the Examples section below. (Bottom) Independent-samples t-test summary for 72 hour data.

### DETAILED DESCRIPTION

The present disclosure relates to normobaric conditions, and in particular the co-administration of non-anesthetic protective gases, and liquid anesthetic agents, to provide anesthesia as well as organ-protection. The organ-protection may protect, but is not restricted to, the brain, spinal cord, kidney, liver, lungs, heart and other tissues against damage resulting for example from regional or systemic hypoxemia, inflammation and the subsequent leakage of vessels, anesthetics diminishing autoregulation of blood pressure in the brain, and detrimental effects of by-products (e.g. formaldehyde) created when anesthetics react with chemical absorbers used in anesthetic circuits. In an optional embodiment, the organ-protection is focused on protecting organs of a subject's torso, such as the kidney, liver and heart (torso, or non-neural, organ protection). The non-anesthetic protective gases may provide some protection against the side effects of the liquid anesthetic agents, and also against the harmful effects of oxygen deprivation.

As used herein, the term “liquid anesthetic agent” refers to an anesthetic medicament provided (i.e. distributed and/or administered) in the form of a liquid, such as an aqueous solution, or lipid emulsion. Examples of liquid anesthetic agents include, but are not limited to, medicaments distributed in the form of a liquid, and administered in the form of a vapor. For example, sevoflurane, isoflurane, and desflurane are typically distributed as a liquid, and then vaporized to a vapor state (for example by heating) for administration to a patient via inhalation. The subset of liquid anesthetic agents that are vaporized to a vapor state for administration to a patient may also be referred to as “anesthetic vapors”. Further examples of liquid anesthetic agents include, but are not limited to, medicaments distributed in the form of a solid (including bound or encapsulated in a solid), and administered in the form of a liquid, for example by intravenous injection. For example, thiopental is distributed as a powder and is then made up in aqueous solution for administration to a patient. Further examples of liquid anesthetic agents include anesthetic medicaments both distributed and administered in the form of a liquid, such as buprenorphine, barbiturate, propofol, ketamine, hypnomidate, dexmedetomidine.

The term liquid anesthetic agent excludes anesthetic agents distributed and administered in the form of a gas. Examples of anesthetic agents that are distributed and administered in the form of a gas include xenon, and nitrous oxide.

As used herein, the term “non-anesthetic protective gas” refers to gases that provide organ protection, without the ability to provide anesthesia effects under normobaric conditions, such as a sedation, sleep or pain control effects. Examples of non-anesthetic protective gases include the noble gases, with the exception of xenon, which is known to provide anesthesia at normobaric pressure. Gases that can provide anesthetic effects, such as xenon and nitrous oxide, are excluded from the definition of non-anesthetic protective gases.

As used herein, the term “normobaric” refers to a pressure of about 1 atm (normal air pressure at sea level; approximately 0.1 MPa). It is understood that normobaric conditions will vary according to location and therefore “about” 1 atm will be understood to include naturally occurring variations in atmospheric pressures above or below 1 atm.

As used herein, the term “administration under normobaric conditions” means administration to the patient whilst exposed to a normobaric environment, and includes pressures generated by a typical anesthesia machine, within a cardio pulmonary bypass or by an ECMO setup. Typically this means that exogenous pressure is not applied to the environment by a device, for example a pressure chamber.

As used herein, the term “organ-protection” means protecting an organ or group of organs, such as a brain, spinal cord, kidney etc., against harmful processes such as hypoxemia, impact trauma etc.

As used herein, the term “subject” includes both humans and non-human animals such as mammals, and includes living beings as well as beings that have been declared brain-dead (e.g. prior to organ donation). In some examples, the term “subject” includes a whole body, or part of a body, such as an organ. For example, an organ, after removal from an organ donor and prior to transplant to a recipient, may be considered a “subject”. In some examples, the term “subject” may refer to an adult. In some examples, the term “subject” may alternatively or additionally refer to a child. In some examples, the term “subject” may alternatively or additionally refer to an infant. In some examples, the term “subject” may alternatively or additionally refer to a neonate.

The present disclosure provides the use of a non-anesthetic protective gas in combination with a liquid anesthetic agent for co-administration at normobaric pressure, to provide organ-protection. The co-administration is typically pre-administration, concurrent administration, and/or post-administration, in which the non-anesthetic protective gas and liquid anesthetic agent are present in the subject in effective amounts to provide anesthetic effect and organ protection, typically in a synergistic manner.

As noted above, non-anesthetic protective gases include the noble gases, excluding xenon (which has anesthetic effects). The noble gases are those elements found under Group 18 of the periodic table. The currently known noble gases are helium, neon, argon, krypton, xenon and radon.

It has been found that administering argon under normobaric conditions provides significant neuroprotective effects. Furthermore, administration of noble gases other than xenon, e.g. argon, at high inspiratory concentrations, does not provide anesthetic effects under normobaric conditions. It is believed that this is the first disclosure of i) the synergistic organ-protective effect of the co-administration of non-anesthetic protective gases and liquid anesthetics, and ii) the ability of such a combination to overcome the inability of most noble gases to provide anesthesia under normobaric conditions. Xenon at 71% has a capacity to provide anesthesia.

The non-anesthetic protective gases also include hydrogen sulfide. Certain clinical effects of hydrogen sulfide are discussed in Kimura and Kimura, 2004; Elrod et al., 2007; Sivarajah et al., 2009; Chen et al., 2010; King and Lefer, 2011; Marutani et al., 2012; Liu et al., 2014; Zhong, 2010; Wang et al., 2014; Gong et al., 2010; and Tang et al, 2013.

The non-anesthetic protective gases may be administered to a patient as part of a gas mixture that includes oxygen. For example, the concentration for argon may be 79% or less, 70% or less, 60% or less, or 50% or less in oxygen. In some examples, the concentration for argon may be between about 79% and about 1%. For further example, the concentration for hydrogen sulfide may be less than about 1000 ppm, or less than about 500 ppm.

In some examples the liquid anesthetic agent may be administered in the form of a vapor, for use in treatment of a subject in need thereof by administration by inhalation or simulated inhalation. Examples of anesthetic vapors include sevoflurane, isoflurane and desflurane.

As used herein, the term “simulated inhalation” refers to those situations where a patient is or may be unable to achieve sufficient gas exchange using their lungs, and is therefore placed on a heart-lung machine (also known as a cardiopulmonary bypass machine), extracorporeal membrane oxygenation (ECMO) or similar device where a membrane based device allows for the exchange for example of gases and vapors between the blood stream and a gas mixture supplied to the other side of the membrane. In such circumstances, the non-anesthetic protective gas is administered to the oxygenator of the heart-lung machine, which simulates the function of the patient's lungs in allowing oxygen (and the protective gas/anesthetic vapor) to diffuse into (and carbon dioxide to diffuse out of) blood drawn from the patient. The oxygen-enriched blood is then pumped back to the patient. In such examples, the liquid anesthetic agent may be also be administered via simulated inhalation, or may be administered intravenously.

The present disclosure also provides a method of providing organ-protection comprising the use of a non-anesthetic protective gas in at least 21% oxygen inhaled and a liquid anesthetic agent, such as propofol, administered to a subject in need thereof over an intravenous route to achieve anesthesia. Other examples of liquid anesthetic agents suitable for administration via the intravenous route include: ketamine, hypnomidate and dexmedetomidine.

In some examples, the non-anesthetic protective gas and liquid anesthetic agent are administered under normothermic conditions. For example, the non-anesthetic protective gas and liquid anesthetic agent may be administered at room temperature, without inducing hypothermia.

In some examples, the non-anesthetic protective gas and liquid anesthetic agent may be administered in the absence of any anesthetic gases, such as xenon or nitrous oxide. For example, although trace amounts of xenon and nitrous oxide may be present in the ambient air, no xenon or nitrous oxide are actively administered to the patient.

The methods described herein may be carried out with an apparatus as disclosed in U.S. patent publication no. US 20100031961 A1, entitled “Retention of Noble Gases in The Exhaled Air of Ventilated Patients By Membrane Separation”. This publication describes the processing of gas mixtures, in particular, of respiration gases for ventilated patients. The processing according to the disclosure relates, in particular, to the use of selective gas separation membranes for the retention of noble gases in the exhaled air of ventilated patients. The gas separation membrane is a separator, which is integrated in a ventilator or an anesthesia machine. The separation membrane separates the noble gases from the remainder of the residual of the exhaled air by selectively retaining sequestering or exhausting the noble gases as desired. Thus, it is possible to provide a ventilator which enables the application of non-anesthetic protective gases, in particular, but not limited to, argon preferably with low loss and as simple as possible.

The methods may also be carried out with an apparatus as disclosed in WO 2007006377 A1, entitled “Device and Method for Preparing Gas Mixtures”, also published as US20070017516, which is incorporated by reference herein in its entirety.

In one embodiment, the non-anesthetic protective gas delivery described above refers to the combined application of the non-anesthetic protective gas in oxygen with an anesthetic vapor. This is typically achieved by inhalation of the non-anesthetic protective gas and the liquid anesthetic as a vapor.

In another embodiment, the non-anesthetic protective gas delivery comprises the administration of a non-anesthetic protective gas, which is stored in pressurized containers until administration to the subject, and the simultaneous or sequential application of a liquid anesthetic agent such as propofol by an intravenous route.

In a further embodiment, the non-anesthetic protective gas/anesthetic vapor delivery device comprises a heart-lung machine. Operation of such a machine has been briefly described above.

In other examples, the non-anesthetic protective gas may be encapsulated or bound in a compound. For example, hydrogen sulfide may be bound to sodium as sodium hydrogen sulphite, a solid that can be solved in water that releases hydrogen sulfide when in the body. This form of hydrogen sulfide can be administered for example in the blood stream, orally or rectally.

As noted above, in the present disclosure, the term “subject” may in some examples refer to an organ outside of a living body. For example, an organ may be removed from an organ donor for transplant to a recipient. When the organ is in transit (i.e. after removal and prior to transplant), a liquid anesthetic agent and a non-anesthetic protective gas may be administered to the organ. For example, the organ may be stored under an atmosphere that includes an anesthetic vapor and a non-anesthetic protective gas. In such examples, the liquid anesthetic agent may be administered to provide synergistic organ protective effects with the non-anesthetic protective gas.

The embodiments described in this disclosure are representative of the subject matter, which is broadly contemplated by the present invention. The scope of the present invention fully encompasses other embodiments, and the scope of the claims should not be limited by the embodiments set forth in the description and examples, but should be given the broadest interpretation consistent with the description as a whole.

### EXAMPLES

### Methods

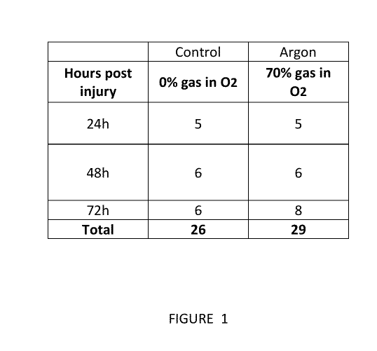

B6.Cg-Tg(Thy1-YFP)16Jrs/J mice: Thy1-YFP mice were exposed to a vapor anesthetic agent (Isofluorane or Sevofluorane), in an anesthetic induction chamber. Animals were temperature probed, sterile eye lubricant was applied to prevent corneal damage, and an incision was made adjacent to the orbital ridge. The left orbit was exposed, and the lacrimal gland gently resected and covered with saline soaked gauze. The superior extraocular muscles were then used to rotate the eye and expose the optic nerve. Using microscissors, the dura was resected, and the optic nerve was transected approximately 0.5 mm from the posterior aspect of the eye bulb. Preservation of blood supply was evaluated through the dissecting microscope focused through a dilated pupil and a coverslip. The skin was closed with suture clips, analgesics (buprenorphine in saline) administered, and the mouse was transferred to an Argon (70%)/Oxygen (30%) perfused air chamber on a warming mat. Mice were monitored and housed in ventilated racks for 24 or 72 hours post-injury. Control mice were exposed to normal atmospheric conditions post-injury (See FIG. 1). Internal surgical controls were the uninjured retina of the contralateral eye.

Retinal Dissection, dissociation, and staining of cells: Mice were lightly anesthetized with Isofluorane and killed via cervical dislocation. Eyes were rapidly removed and placed in ice cold Hank's Balanced Salt Solution (HBSS). A single puncture to the conjunctiva was made, and corneas removed by microdissection. The ciliary epithelium and lens were then resected, and vitreous humor was visualized using triamcinolone acetonide solution to aid in removal by aspiration and microdissection. Free floating retinas were then transferred to fresh HBSS on ice, then transferred to 1 ml of enzyme solution containing Trypsin (MACS—Miltenyi Biotec). Following a 15 minute incubation at 37 degrees C. with periodic agitation, retinas were dissociated with fire polished pipettes and centrifuged. Pellets were washed in Annexin V binding buffer, and centrifuged. Pellets were the re-dissociated in DNAse-containing binding buffer, and stained for Annexin V-PeCy7 probe. Cells were washed, cell strained (40 um), and analyzed using a BD FacsAria III flow cytometer.

Flow cytometry and data analysis: Compensation and gating was established using unstained, YFP-only, Annexin V-only, and double-labeled cell samples. Dead cell assays (not shown) were also performed using propidium iodide and 7-AAD DNA intercalating dyes. Forward and side scatter gating were used to exclude debris, and YFP-positive cells were gated and used as the reference population. Quadrant analysis was used to identify and quantify Annexin V labeling within YFP expressing cells across groups. A within-subjects comparison was performed by comparing injured and uninjured retinas from the same animal. Difference scores (uninjured—injured Annexin V cell counts) were produced in order to normalize Annexin V background labeling in right eyes. All data are represented as means +/−1.0 standard error of the mean.

Retinal histology: Eyes were enucleated, corneas and lenses removed, and immersed in ice cold 4% paraformaldehyde for 25 minutes. Following 30% sucrose cryoprotection, eyes were introduced to OCT medium, embedded, cut on a cryostat at 14 microns and mounted onto slides. Topro-3 iodide and DRAQ5 nuclear labels were used to evaluate retinal architecture and integrity. YFP and cleaved caspase-3 immunolabeling were used to evaluate early induction of apoptosis in the ganglion cell layer of Thy-1 reporter animals. All imaging was performed on a Zeiss LSM 710 confocal microscope.

### Results

Annexin V-PeCy7 evaluation of early apoptosis in injured retinal ganglion cells reveal a neuroprotective role for argon post-injury treatment.

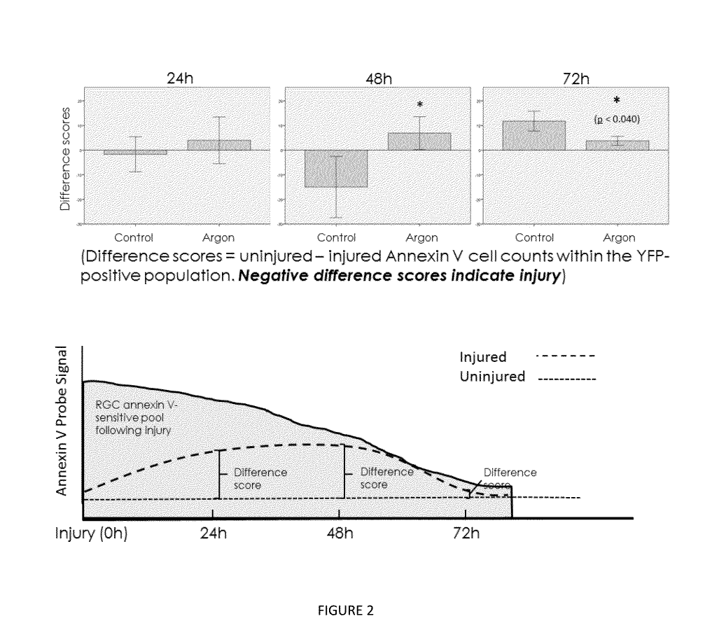

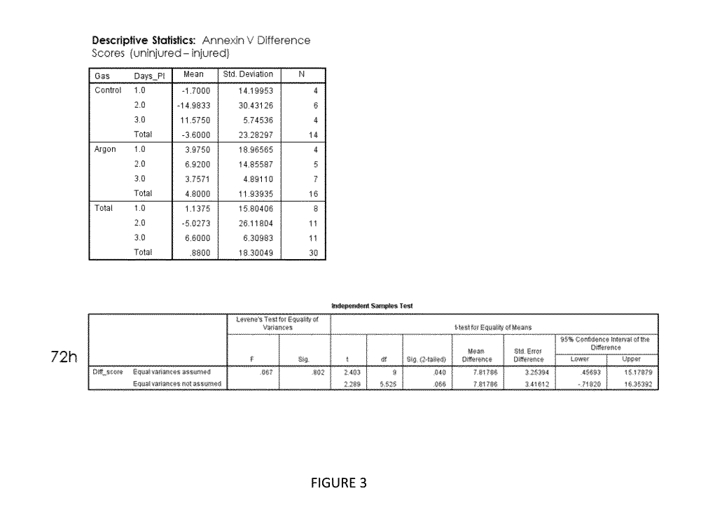

To evaluate the early apoptotic events that follow neural injury, dissociated retinas were probed from Thy-1-YFP reporter mice that underwent unilateral retinal optic nerve axotomy, with a selection of Annexin V dyes. These dyes bind with inner leaflet exposure/externalization of phosphatidylserine that accompanies early apoptosis. Comparing uninjured and injured retinas using flow cytometric counts (500,000 to 1,000,000 events per run) was accomplished by subtracting injured, Annexin V-positive counts from those obtained from uninjured retinas, within the YFP-positive population. At 24 h, a slight negative mean difference score in controls indicated that early Annexin V binding events are evident, reflecting an onset of RGC injury (FIGS. 2 and 3). In contrast to control retinas, injured retinas exposed to argon exhibit a lower level of Annexin V sensitivity, as indicated by a positive difference score, suggesting that the neuroprotective function of argon is present at this time. By 48 h, a strong negative difference score in control retinas indicate the peak of Annexin V binding in injured retinas. In contrast to control retinas, injured retinas exposed to Argon maintain a strong resistance to Annexin V binding, suggesting that the peak of neuroprotection effect size resides around 48 h post-injury. At 72 h post-injury, positive difference scores in control retinas is evident. This observation likely represents a dominance of background labeling that occurs due to sample preparation this time, and a reduction in available RGCs that may be sensitive to Annexin V binding as they have already progressed through early apoptosis.

### Discussion

These data show that argon, when administered in combination with liquid anesthetics, provides a neuroprotective effect within the central nervous system. It is expected that this effect will occur when argon is administered concurrently with liquid anesthetics, prior to liquid anesthetics, and/or subsequent to liquid anesthetics.

It is believed from these data that a larger proportion of RGCs manage to survive over the long term. It is expected that later time points will reveal a sustained population of surviving RGCs following argon exposure.

### REFERENCES

Kundra, P., Vinodhadevi, V., & Arimanickam, G. (2011). Sevoflurane-induced arrhythmia in an adult and a child. Journal of Anaesthesiology, Clinical Pharmacology, 27(2), 269-71. doi:10.4103/0970-9185.81844
Regueiro-Purriños, M., Fernández-Vázquez, F., de Prado, A. P., Altónaga, J. R., Cuellas-Ramón, C., Ajenjo-Silverio, J. M., . . . Gonzalo-Orden, J. M. (2011). Ventricular arrhythmias and mortality associated with isoflurane and sevoflurane in a porcine model of myocardial infarction. Journal of the American Association for Laboratory Animal Science: JAALAS, 50(1), 73-8.
Zhong, G.-Z. (2010). Hydrogen Sulfide-a potent multichannel anti-arrhythmic drug. Journal of Cardiovascular Disease Research, 1(1), 37-9. doi:10.4103/0975-3583.59984
Tétrault, S., Chever, O., Sik, A., & Amzica, F. (2008). Opening of the blood-brain barrier during isoflurane anaesthesia. The European Journal of Neuroscience, 28(7), 1330-41. doi:10.1111/j.1460-9568.2008.06443.x
Hu, N., Guo, D., Wang, H., Xie, K., Wang, C., Li, Y., . . . Wang, G. (2014). Involvement of the blood-brain barrier opening in cognitive decline in aged rats following orthopedic surgery and high concentration of sevoflurane inhalation. Brain Research, 1551, 13-24. doi:10.1016/j. brainres.2014.01.015
Wang, Y., Jia, J., Ao, G., Hu, L., Liu, H., Xiao, Y., . . . Cheng, J. (2014). Hydrogen sulfide protects blood-brain barrier integrity following cerebral ischemia. Journal of Neurochemistry, 129(5), 827-38. doi:10.1111/jnc.12695
Gong, Q.-H., Wang, Q., Pan, L.-L., Liu, X.-H., Huang, H., & Zhu, Y.-Z. (2010). Hydrogen sulfide attenuates lipopolysaccharide-induced cognitive impairment: a pro-inflammatory pathway in rats. Pharmacology, Biochemistry, and Behavior, 96(1), 52-8. doi:10.1016/j.pbb.2010.04.006
Liu, Y., Ye, Z., Yang, H., Zhou, L., Fan, D., He, S., & Chui, D. (2010). Disturbances of soluble Nethylmaleimide-sensitive factor attachment proteins in hippocampal synaptosomes contribute to cognitive impairment after repetitive formaldehyde inhalation in male rats. Neuroscience, 169(3), 1248-5 doi:10.1016/j.neuroscience.2010.05.061
Tong, Z., Han, C., Luo, W., Wang, X., Li, H., Luo, H., . . . He, R. (2013). Accumulated hippocampal formaldehyde induces age-dependent memory decline. Age (Dordrecht, Netherlands), 35(3), 583-96. doi:10.1007/s11357-012-9388-8
Li, X., Zhang, K.-Y., Zhang, P., Chen, L.-X., Wang, L., Xie, M., . . . Tang, X.-Q. (2014). Hydrogen sulfide inhibits formaldehyde-induced endoplasmic reticulum stress in PC12 cells by upregulation of SIRT-1. PloS One, 9(2), e89856. doi:10.1371/journal.pone.0089856
Kimura, Y., & Kimura, H. (2004). Hydrogen sulfide protects neurons from oxidative stress. FASEB Journal: Official Publication of the Federation of American Societies for Experimental Biology, 18(10), 1165-7. doi:10.1096/fj.04-1815fje
Elrod, J. W., Calvert, J. W., Morrison, J., Doeller, J. E., Kraus, D. W., Tao, L., . . . Lefer, D. J. (2007). Hydrogen sulfide attenuates myocardial ischemia-reperfusion injury by preservation of mitochondrial function. Proceedings of the National Academy of Sciences of the United States of America, 104(39), 15560-5. doi:10.1073/pnas.0705891104
Sivarajah, A., Collino, M., Yasin, M., Benetti, E., Gallicchio, M., Mazzon, E., . . . Thiemermann, C. (2009). Anti-apoptotic and anti-inflammatory effects of hydrogen sulfide in a rat model of regional myocardial I/R. Shock (Augusta, Ga.), 31(3), 267-74. doi:10.1097/SHK.0b013e318180ff89
Chen, Y., Liu, Z., & Xie, X. (2010). Hydrogen sulphide attenuates renal and cardiac injury after total hepatic ischemia and reperfusion. The Journal of Surgical Research, 164(2), e305-13. doi:10.1016/j.jss.2010.08.010
King, A. L., & Lefer, D. J. (2011). Cytoprotective actions of hydrogen sulfide in ischaemia-reperfusion injury. Experimental Physiology, 96(9), 840-6. doi:10.1113/expphysio1.2011.059725
Marutani, E., Kosugi, S., Khatri, A., Nguyen, R., Atochin, D. N., Kida, K., . . . Van Leyen, K. (2012). A novel hydrogen sulfide-releasing N-methyl-D-aspartate receptor antagonist prevents ischemic neuronal death. The Journal of Biological Chemistry, 287(38), 32124-35. doi:10.1074/jbc.M112.374124
Liu, D., Wang, Z., Zhan, J., Zhang, Q., Wang, J., Zhang, Q., . . . Hao, A. (2014). Hydrogen sulfide promotes proliferation and neuronal differentiation of neural stem cells and protects hypoxia-induced decrease in hippocampal neurogenesis. Pharmacology, Biochemistry, and Behavior, 116, 55-63. doi:10.1016/j.pbb.2013.11.009

1. A method of providing anesthesia and organ-protection to a subject in need thereof, comprising co-administering to the subject a non-anesthetic protective gas in an amount effect to provide organ protection, and a liquid anesthetic agent in an amount effective to provide anesthesia, at normobaric conditions.

2. The method according to claim 1 wherein the non-anesthetic protective gas is helium, neon, argon, krypton, or radon.

3. The method according to claim 2 wherein the non-anesthetic protective gas is argon.

4. The method according to claim 2, wherein the argon is administered at a concentration of between 1% and 79%.

5. The method according to claim 1 wherein the non-anesthetic protective gas is hydrogen sulfide.

6. The method according to claim 5 wherein hydrogen sulfide is administered at a level of less than 1000 ppm.

7. The method according to claim 1 wherein the liquid anesthetic agent is administered in liquid form.

8. The method according to claim 7 wherein the liquid anesthetic agent is thiopental, propofol, ketamine, hypnomidate, barbiturate, buprenorphine, or dexmedetomidine.

9. The method according to claim 7 wherein the liquid anesthetic agent is administered intravenously.

10. The method according to claim 1 wherein the liquid anesthetic agent is administered in a vapor state.

11. The method according to claim 10 wherein the liquid anesthetic agent is sevoflurane, isoflurane or desflurane.

12. The method according to claim 11 wherein the liquid anesthetic agent is sevoflurane.

13. The method according to claim 10 wherein the non-anesthetic protective gas and the liquid anesthetic agent are administered to the subject by a membrane-based device.

14. The method according to claim 10, wherein the non-anesthetic protective gas and the liquid anesthetic agent are administered to the subject by a ventilator.

15. The method according to claim 14 wherein the ventilator comprises one or more gas separation membranes that selectively retains, sequesters or exhausts the non-anesthetic protective gas from the air exhaled by the subject, and optionally i) when the non-anesthetic protective gas is retained it is available for further use on the patient and ii) when the non-anesthetic protective gas is sequestered or exhausted, it is subsequently recaptured for future use.

16. The method according claim 1 wherein administration of the non-anesthetic protective gas in combination with the liquid anesthetic agent reduces damage to the brain, the spinal cord, the kidney, the liver and/or the heart.

17. The method according to claim 1 wherein administration of the non-anesthetic protective gas in combination with the liquid anesthetic agent reduces damage resulting from hypoglycemia, hypoxemia, hypoxia, ischemia, cerebral edema or axotomy.

18. The method according to claim 1 wherein the non-anesthetic protective gas and the liquid anesthetic agent are administered concurrently or sequentially.

19. The method according to claim 1 wherein the liquid anesthetic agent and non-anesthetic protective gas are administered to a patient that is intubated and ventilated for stroke, is undergoing surgery, including cardiac surgery, neurosurgery optionally general surgery or trauma surgery, optionally trauma surgery to treat impact trauma, such as traumatic CNS injury (brain injury or spinal cord injury) or traumatic injury to organs in the torso.

20. The method according to claim 1, wherein the non-anesthetic protective gas and the liquid anesthetic agent are administered in the absence of xenon.

21. The method according to claim 1, wherein the non-anesthetic protective gas and the liquid anesthetic agent are administered in the absence of nitrous oxide.

22. The method according to claim 1, wherein the non-anesthetic protective gas and liquid anesthetic agent are administered under normothermic conditions.

23. A use of a non-anesthetic protective gas in combination with a liquid anesthetic agent in an amount effective to provide anesthesia, at normobaric conditions, for providing anesthesia and organ-protection to a subject in need thereof, optionally wherein the non-anesthetic protective gas is argon, optionally wherein the liquid anesthetic agent is sevoflurane, isoflurane or desflurane.

24. A method of providing organ-protection to a subject in need thereof, comprising co-administering to the subject a non-anesthetic protective gas and a liquid anesthetic agent, in amounts effective to provide organ protection, at normobaric conditions.
